(19) United States
(12) Patent Application Publication
(10) Pub. No.: US 2023/0261874 A1
(43) Pub. Date: Aug. 17, 2023

(54) Method and System to Generate Personalized Digital Code from Unique Genome Pattern for Use in Identification and Application on Blockchain

(71) Applicant: Isaac Kise Lee, San Diego, CA (US)

(72) Inventor: Isaac Kise Lee, San Diego, CA (US)

(21) Appl. No.: 17/673,844

(22) Filed: Feb. 17, 2022

(51) Int. Cl. H04L 9/32; G16B 30/20

(52) U.S. Cl. CPC H04L 9/3226; G16B 30/20; H04L 2209/38

(57) ABSTRACT

This disclosure relates generally to a systematic method directed towards methods and systems for developing a personalized and secure digital code from individual genome data and nucleic acid analysis. Unique sequence and combinations from common single nucleotide polymorphisms (SNPs) will consist of the markers needed to create a personalized code combination. The final output can be utilized to create various modes of identification on the decentralized blockchain network, personalized service and products offering allowing for a robust and novel method for identification and personalization based on an individual's genomic fingerprint and biological characteristics.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

### Field of the Invention

The invention relates in part to products and processes for developing a biologically unique digital identification code (numerical or alphabetical) from an individual's genome sequence for use in decentralized blockchain identification and application.

### Description of the Related Art

There is approximately 7.8 billion people in the world and a large majority use computers, smartphones, internet, and internet-connected devices (IoTs). According to a US survey in 2020, 87 percent of individuals have access to a computer in their households and about 5 billion active internet users in the world today. This number is expected to grow exponentially with the recent revolution of IoT, Web3.0 and Metaverse applications. Traditional identity and personalization systems in use today are often fragmented, insecure, and exclusive. The various data associated with the current fragmented and exclusive identification method is difficult to track, combine and co-utilize in the many applications in a secure, private, and effective manner. Blockchain technology enabled identification method could potentially allow for more secure management and storage of digital identities by providing unified, interoperable, and tamper-proof infrastructure with key benefits to enterprises, users, and IoT management systems. Having a unique, personalized identification code allows the individual to safely identify themselves without having to reveal other personal information to other parties with the added benefit of the code being truly unique to that individual.

Individual identity and personalization are integral to a functioning society and economy. Having a safe, secure, and robust way to identify and personalize ourselves and our possessions is becoming increasingly crucial in today's digital society and global markets. At its most basic level, identity is a collection of claims about a person, place or thing. For people, this usually consists of first and last name, date of birth, nationality, and some form of national identifier such as one's passport number, social security number (SSN), driving license, etc. These data points are issued by centralized entities (governments) and are stored in centralized databases (central government servers). A digital identity arises organically from the use of personal information on the web and from shadow data created by the individual's actions online. More robust identity and personalization management systems could be used to eradicate current identity issues such as inaccessibility, data insecurity, and fraudulent identities. Security and identity are complex and ever-evolving issues for enterprise and government systems alike. Blockchain-based solutions could provide exceptional utility in solving issues common in current identity and digital systems. The blockchain technology allows for users to create and manage digital identities through the combination of decentralized identifiers, identity management and embedded encryption. However, current blockchain based identification methods rely on randomly generated digital code that is not unique to the individual. The genome fingerprint based personalized digital code is unique to each individual and could potentially be used to verify ownership of identity even in the case of when a private key or passcode is not available for the associated data.

The human genome holds roughly 3 billion base pairs. Each person's genome sequence holds a unique combination formulated from random recombination from the parent's genome during fertilization. Half of the individual genome is inherited from the father and the other half from the mother. The genetic makeup is composed of four nucleotides (ACGT) and shares 99% similarities among all people. The sequence of the genome can be digitally expressed by comparing the reference genome sequence position and alleles to that of an individual sequence. Despite the long sequence, only 0.1% of the genome accounts for all the genetic variation found in every individual. This indicates that there are potentially more than three million base pair differences between two different individuals' genome. Most genetic variations are in the form of single nucleotide polymorphisms (SNPs), which have been extensively studied by many acclaimed scientists after the completion of the Human Genome Project (HGP) in 2003. More than 20 millions of these variations have been mapped and analyzed among different ethnicities and nationalities. Most of these specific variations are rather rare and show an individual and population-specific occurrence, but some fraction of these variations arises very frequently in every population studied. These high allele frequency (close to 50%) pan-ethnic common SNPs are considered to be natural results from evolution history and are not associated with any known phenotype or disease. Most of these variations are bi-allelic, which means only two choices of nucleotides are observed at a given specific site. The patterns of these high allele frequency bi-allelic SNP variations can be used to generate a personalized genetic code that can potentially identify and track individuals for use in forensics, such as the CODES method that uses fragment length polymorphisms. The unique combination pattern from SNPs can act as a unique identifier much like an individual's fingerprint. Authentication of one's identity can only be possible when the pattern of the genetic variation matches that of a known identification in a database, allowing for sensitive personal identification information to be encrypted in anonymous identification purposes.

The current application has the novel feature of employing the bi-allelic, high frequency, pan-ethnic common SNP variations in the genome to prepare and create a unique genomic pattern akin to fingerprint authentication (Genome Fingerprinting). This information can provide accurate and powerful means to identify and distinguish individual people in digital format by simply decoding the digital code without having an identifiable personal information. The current procedure aims to imply a panel of selective, universal, high allele frequency bi-allele human SNP markers to efficiently generate a unique combination code that can be used in digital identification and tracking.

Thus it has been noted, to Applicant's knowledge, none of these prior art methods is entirely suitable to meet these needs and is cumbersome. Therefore, there is a need in the prior art in which the inconveniences, as mentioned earlier, difficulties, and grooming problems are eliminated for all practical purposes. Thus, the present invention provides such a method, and the overall combination of these features is nowhere disclosed in the prior art cited above, which appears to represent the general art in this area, although it is not intended to be an all-inclusive listing of pertinent prior art patents.

### SUMMARY

In light of the disadvantages of the prior art, the following summary is provided to facilitate an understanding of some of the innovative features unique to the present invention and is not intended to be a complete description. A full appreciation of the various aspects of the invention can be gained by taking the entire specification, claims, drawings, and abstract as a whole.

The present subject matter relates generally to method and system to generate personalized digital code from unique genome pattern for use in identification and application on blockchain.

The invention employs a patterns of high allele frequency bi-allelic SNP variations to generate a personalized genetic code that can potentially identify and track individuals for use in forensics, such as the CODES method that uses fragment length polymorphisms.

Another embodiment of the invention is that unique combination pattern from SNPs can act as a unique identifier much like an individual's fingerprint.

Another embodiment of the invention is that the bi-allelic, high frequency, pan-ethnic common SNP variations in the genome is used to prepare and create a unique genomic pattern akin to fingerprint authentication termed as Genome Fingerprinting.

It is accordingly an object of the invention to provide a provide accurate and powerful means to identify and distinguish individual people in digital format by simply decoding the digital code without having an identifiable personal information.

Another novel feature of the invention is to employ a panel of selective, universal, high allele frequency bi-allele human SNP markers to efficiently generate a unique combination code that can be used in digital identification and tracking.

Although the invention is illustrated and described herein as embodied in as to generate personalized digital code from unique Genome, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

Other objects, features and advantages of the present invention will be apparent from the accompanying drawing and from the detailed description which follows.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### Brief Description of Drawing

non-limiting and non-exhaustive embodiments of the present invention are described with reference to the following drawings. The system and method of the present invention will now be described with reference to the accompanying flow chart drawing figure, in which:

FIG. 1 shows an example of species code illustrating embodiment of proposed invention.

FIG. 2 illustrates a Gender code with possible outcomes for both natural and virtual birth illustrating embodiment of proposed invention.

FIG. 3 Illustrates a possible outcomes of each genetic markers' digital code including three possible combinations plus no result calls illustrating embodiment of proposed invention.

FIG. 4 Illustrates a possible outcomes of each genetic markers' digital code including three possible combinations plus no result calls illustrating embodiment of proposed invention.

FIG. 5 Illustrates an auxiliary code for genetically identical individuals such as twins and clones illustrating embodiment of proposed invention.

FIG. 6 Illustrates Theoretical combinations codes from the given SNP marker analysis based on three different allele combinations (bi-allele SNP analysis) illustrating embodiment of proposed invention.

FIG. 7 Illustrates an example of a 100 candidate SNP marker selection from a public HapMap and 1000 Genome Sequencing database for digital identification and personalization purpose illustrating embodiment of proposed invention.

FIG. 8 Illustrates an upper table, 24SNP marker selection for the development of a digital identification panel illustrating embodiment of proposed invention.

FIG. 9 Illustrates Digital Identification Code Assignment from the SNP panel data illustrating embodiment of proposed invention.

FIG. 10 Illustrates generating 2D QR Code from the numerical digital genetic identity code from each individual using the UTF-8 encoding illustrating embodiment of proposed invention.

FIG. 11 Illustrates generating 2D QR Code from the alphabetical digital genetic identity code from each individual using the UTF-8 encoding illustrating embodiment of proposed invention.

Skilled artisans will appreciate that elements in the figures are illustrated for simplicity and clarity and have not necessarily been drawn to scale. For example, the dimensions of some of the features in the figures may be exaggerated relative to other elements to improve understanding of embodiments of the present invention. The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The present invention now will be described more fully hereinafter with reference to the accompanying drawings, which form a part hereof, and which show, by way of illustration, specific exemplary embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as methods or devices. Accordingly, the present invention may take the form of an entirely hardware embodiment, an entirely software embodiment or an embodiment combining software and hardware aspects. The following detailed description is, therefore, not to be taken in a limiting sense.

Briefly stated, the present invention is directed towards novel digital identification method comprises of steps that involve selecting genetic markers for identification, assembling information for the unique genetic identification, assigning digital codes for each of the features, creating the digital identification codes, decoding the digital identification code, registering the codes for blockchain, and implementing codes for various applications.

### Digital Identification Format

In order to formulate a digital identification which holds all the necessary information from a wide selection of species of animals and plants with the possibility of a rare event (identical twins or cloning), we propose the following digital identification format for our invention.

Species Code (2-4 digits), Gender Code (1 digit with Natural or Virtual birth separation) (1), Identification Codes (24 digits for numerical, and 12 digits for alphabetical), Auxiliary Code (1 or 2 digits)

### Species Codes

Every animal, plant, bacteria, and virus on earth has genetic makeup (DNA and RNA) vital for growth, reproduction and multiplication. Although the current patent filing is primarily focused on using genetic code in identifying human individuals, the same concept of genetic based blockchain identification can be used in other species for identification, parental linkage, tracking, or management analysis in agricultural businesses. Therefore, species identification code should be included in the digital format. Below is an example of the proposed codes for some of the well-known species of interest. The two-digit combination of the alphabetical code can cover up to 576 (24×24) different species in the code combination. Three (13,824 species) or four (331,776 species) digit species identification code combination may be implemented in the future species application expansion.

### Gender Codes

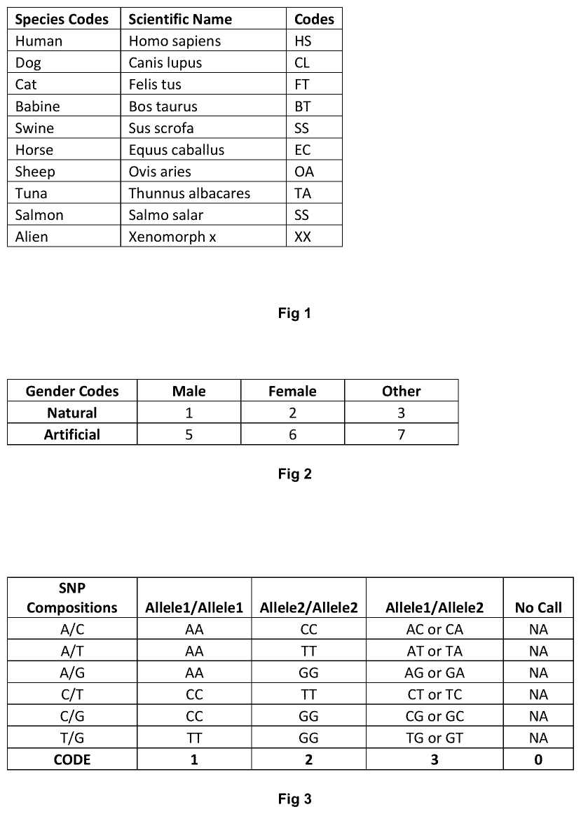

With respect to biological sex, humans are born as either male or female. However, gender classifications are simply not binary so other types (gender neutral, transgender, genetic abnormalities) should be considered in the gender code assignment to cover a wide spectrum of people. Furthermore, we are also exploring the possibility of a non-natural birth (Artificial or Virtual) individual such as cloning or digital creation in some applications, that could be differentiated from real (Natural) individuals to that of artificial or virtual individuals as shown in FIG. 2.

### Individual Genetic Identity Codes

24-digit numerical codes

Because of SNP's mostly bi-allelic nature, each position of the markers has three different possible combinations as summarized in the table below. We can assign a code “1” for the known reference allele, “2” for the alternative allele, and “3” for the mixture of two alleles called heterozygotes. However, there is a possibility of an incomplete or failed sequencing result at a particular site that can stein from either low confidence call or missing call for a certain SNP. The missing calling makes it difficult to assign a correct allele information on the given sites. Therefore, the missing genotypes are labelled “NA” in the sequencing output file and assigned a “0” in the code output. Typically, the standard whole genome sequencing coves roughly 95% of the entire genome region when it is sequenced in 30× read depth (standard Whole Genome Sequencing QC criteria), meaning that there is always a chance that one or more SNPs may not produce a satisfactory result to assign an appropriate code. In order to establish a criteria cutline and a measure of quality control, the minimum data point needed to assign a reliable identity code will be designated to 21 data points. Based on the 24 SNPs needed for human identification code generation used in the patent application, the encoding system can accommodate and produce a valid result even when up to three SNP data points are missing as shown in FIG. 3.

12-digit alphabetical codes

In order to accommodate the limitation of the code number, and shorten the code length, we also invented to convert 24-digit numerical codes to 12-digit alphabetical codes by combining two numerical codes to one alphabetical code.

The numerical code has only four (0-3) options in each digit sites, but alphabetical codes have 16 different options in each digit sites, and gives more options for downstream applications and unitalities.

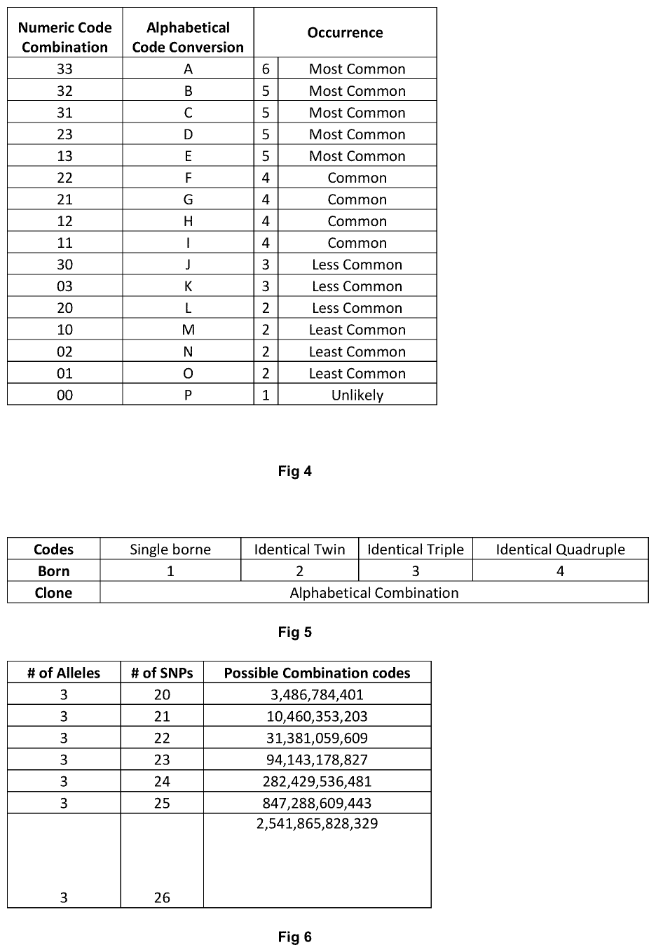

The 12-digit alphabetical codes can be easily converted back to a 24-digit numeric codes, and be able to decode original SNP sequence of each individual people as shown in FIG. 4.

### Auxiliary Codes

One of the drawbacks for using genetic-based identification codes is its inability to separate and distinguish individuals with identical genetic makeups. Such criteria are found in identical twins, triplets, quadruplets, and human clones. Therefore, an auxiliary identification code is needed when two individuals with the same genetic makeup are to be identified. The auxiliary code can also be used to separate real (natural birth) individuals from artificial (virtual birth) individuals that are created in virtual environments through games or other computer algorithms. The auxiliary code can be numeric or alphabetic, both of which can be either single digit or double-digit, depending on the potential number of genetically identical individuals created in a natural (identical twins) or artificial (cloning) environment. For example, we are assigning the number “1” for single born real individuals and “2” for twins with identical genetic makeup. Note that non-identical twins have different genetic makeup and therefore will produce different codes. The auxiliary code is also designed to take into account the very rare event (one out of 280 billion cases when 24 SNPs are being used in the panel) when two unrelated individuals have the same sequencing code by accident as shown in FIG. 5.

### SNP Identity Marker Selection

It is well established that about 0.1% of the genome accounts for all the genetic variations that are found from person to person. This means that there are potentially around three million base pair differences in a particular genome between two non-related individuals. All of these variations in the genome can be used as a fingerprint for patterns when differentiating from other individuals. Therefore, each individual, except identical twins, has a unique genomic fingerprint that solely belongs to that person. Single nucleotide polymorphisms (SNPs) are the most common type of genetic variation, and they are easy to detect with most genotyping and sequencing technology platforms. Extensive research in the human genome project have led to more than 20 million variations being studied in different ethnicity and nationality backgrounds. Most of these variations are quite rare and show individual and population specific distribution patterns, but some of these fractions arise very frequently in every population studied. The majority of these variations are bi-allelic, which means only two choices of nucleotides are observed within a given specific SNP site. The patterns and fingerprints of these high-frequency bi-allelic SNPs have been used in other applications, most notably to identify individuals for forensic purposes.

In order to select the most informative markers for the identification and personalization purposes, SNPs that show a high minor allele frequency (>0.45) from all population studies were selected from HapMap and 1000 genome sequencing database. For example, there are over 4 million SNPs that have been genotyped in the CEU cohort within HapMap, and 218,000 SNPs showed minor allelic frequencies of greater than 45%. The higher the minor allelic frequencies are, the more informative the result will be based on marker combinations present in the genomes. Therefore, fewer markers are required when higher frequency SNP markers are used for testing, analysis, interpretation in the identification and personalization purpose.

### Marker Selection Workflow

Markers are selected from published HapMap and 1000 Genome Sequencing database using the criteria below:
Identify marker sets with high minor allele frequency (e.g. >45%) within the study population
Select only bi-allelic SNP markers with good Hardy-Weinberg distribution
Avoid markers with adjacent known polymorphisms to minimize potential allele drop in sequencing
Find markers that are well separated from each other
Avoid markers in a region containing duplicated sequence motifs
   Number of SNP markers for digital identification code generation

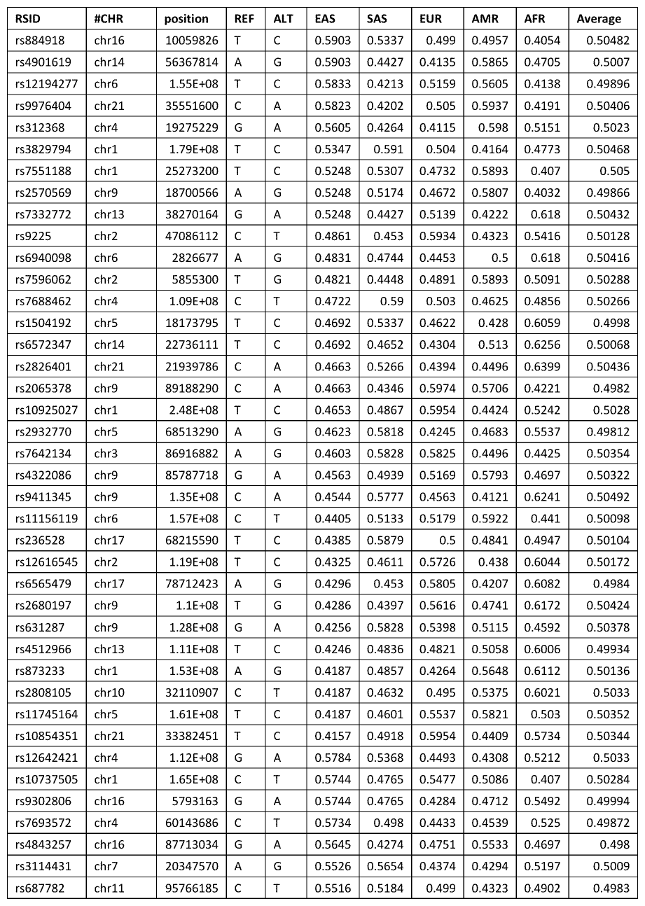

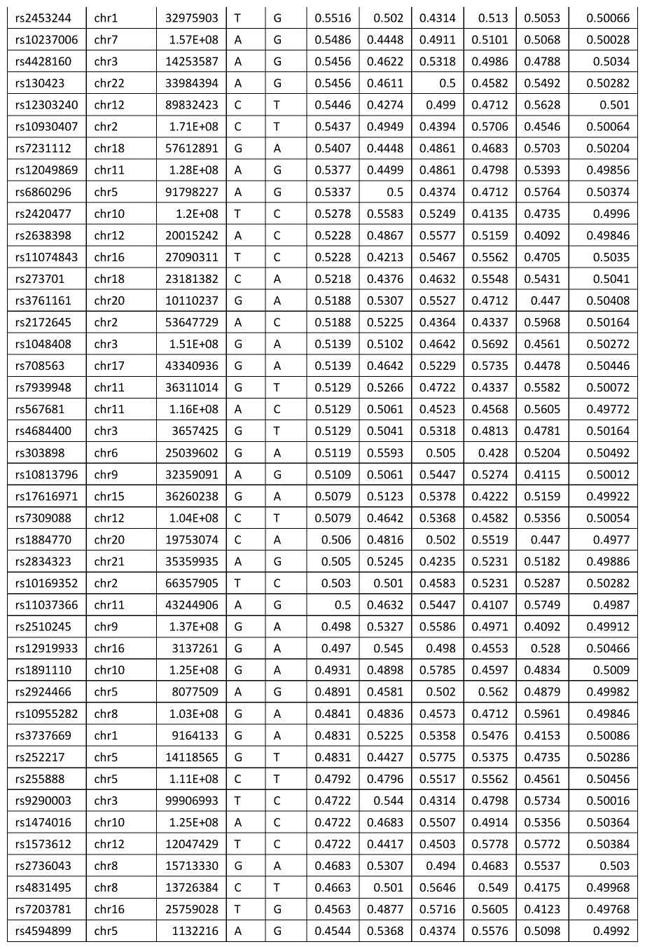

Because each bi-allele SNPs has three different allele combination (Reference, Alternative, Heterozygote), each SNP analysis could generate three distinct identification codes. The possible combination of two unrelated SNP markers could potentially generate 9 (3×3) different codes, and three distinct SNP markers would result in 27 (3×3×3) different combination codes. When taking into account a world population of 7.9 billion people in recent world population survey, a 21 SNPs composition (Over 10 billion possible combination codes) could potentially differentiate all the people in the world. Therefore, we propose to use 24 SNP combinations for the human identification in this patent filing to create sufficient buffer and accommodate for future population growth, as well as virtual creation of artificial individuals as shown in FIG. 6.

Candidate SNP Marker Selection for digital identification

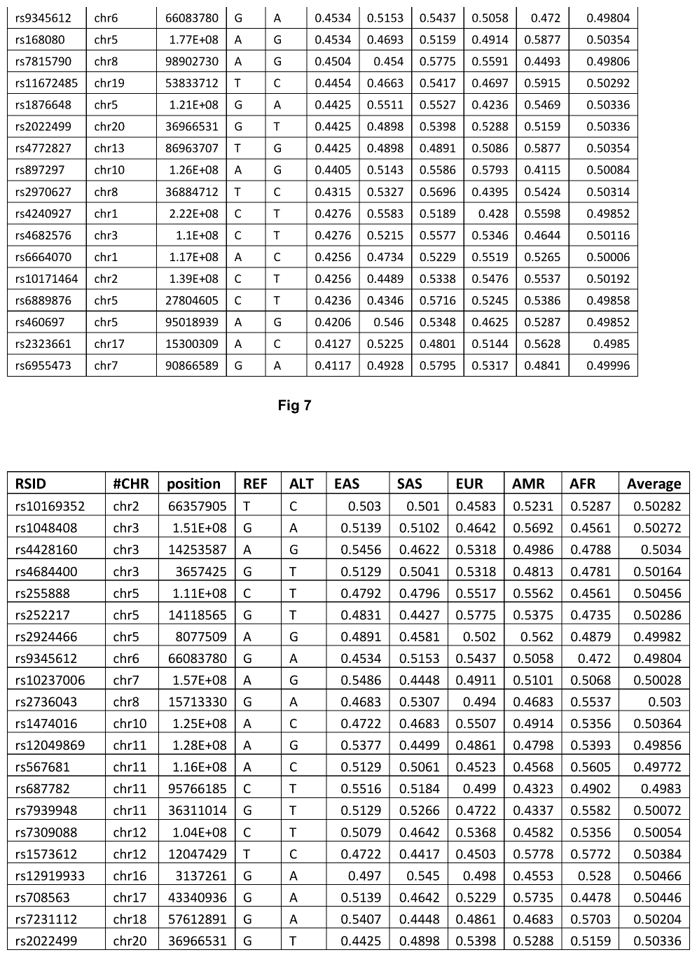

The table below showcases the proposed 100 markers to be used, all of which have an average 50% minor allele frequency ranging from 0.498 to 0.508 in all populations tested by the 1000 genome sequencing project. Therefore, each of these SNPs is expected to be highly present in all populations. The results from any subset of these markers provide enough statistical power to reliably identify and separate every individual from the most genome data available as shown in FIG. 7.

### Digital Identification Panel Design

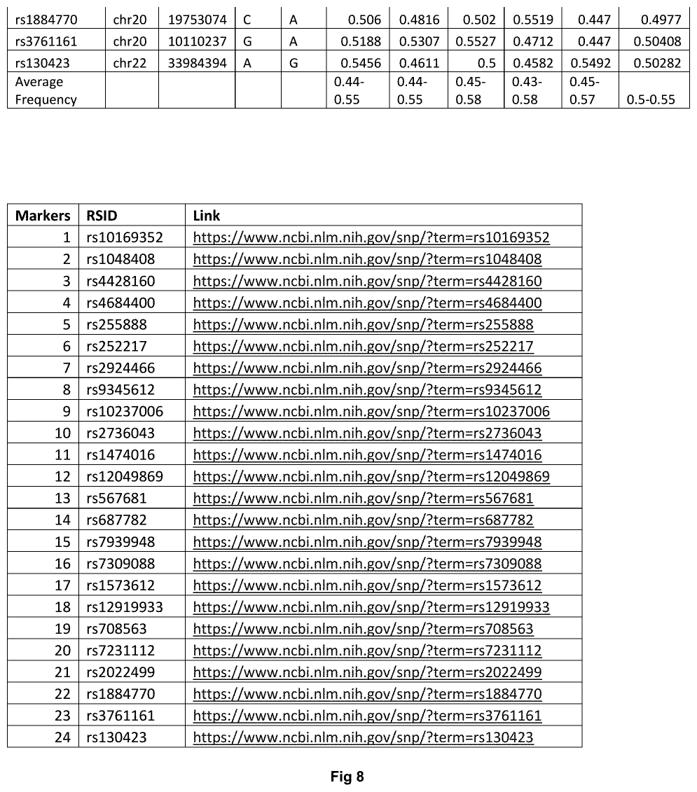

We further selected 24 SNPs from the 100 candidates SNPs for our digital identity panel that showed the least ethnicity differences in all populations tested. The chosen SNPs shows minor allele frequency between 0.43-0.58 in every population tested, allowing for a wide variety of combinations for every individual across all populations. The fixed universal marker set can be used in all individuals in the same species for the digital identification panel as shown in FIG. 8.

Personal Digital Identity Code Assignment from 24 SNPs Panel Collection

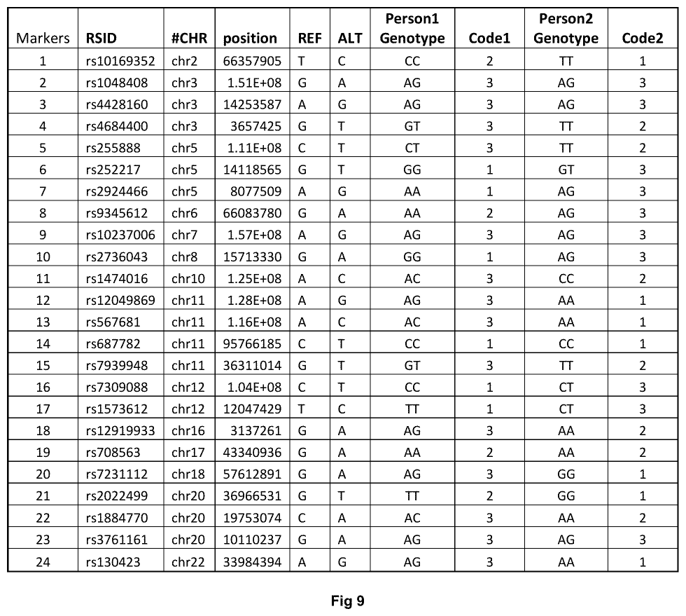

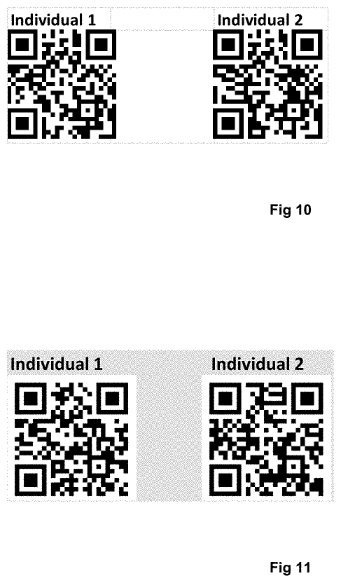

The table below is used to generate a personalized digital code for two real individuals (Person 1 male, and Person 2 female) using selected 24 SNP markers in the panel as shown in FIG. 9.

Generated Personal Genetic Identity Code from Genome Sequences
Individual 1:
   Numerical Codes:233331123133313113232333
   Alphabetical Codes: DACHCACCEDDA
Individual 2:
   Numerical Codes 133223333321112302211231
   Alphabetical Codes: EBDAAGIDNGHC

### Generation of the Personalized Digital ID Code

Format: Species Code (2), Gender Code (1), Identification Codes (24), Auxiliary Code (1). Below are the two people used to generate the digital identification code using the format and composition from this patent. Individual 1 is a male human and single born. Individual 2 is a female human and single born. A 2D barcode can be created from the following code and can be linked to an HTML file or web page to link with additional information.
Individual 1: Human, Male, Single born
   HS,1,233331123133313113232333,1
   HS,1, DACHCACCEDDA,1
Individual 2: Human, Female, Single born
   HS,2,133223333321112302211231,1
   HS,2,EBDAAGIDNGHC,1

It is appreciated that additional advantages, modifications and equivalent embodiments will be apparent to those skilled in the art. Therefore, the invention, in its broader aspects, is not limited to the specific details shown and described herein. Accordingly, various modifications may be made without departing from the spirit or scope of this invention as defined by the appended claims and their equivalents.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The invention claimed is:

1. Methods and systems to using set of bi-allele SNP combinations to generate a biologically unique, personalized digital identity code that can be used in decentralized blockchain for identification and personalized applications.
System as per claim 1, involving a genetic based digital code generation for use in blockchain networks;
System as per claim 1, involving combination of genetic markers used for the purpose of identification and personalization in blockchain networks;
System as per claim 1, involving a method and system to convert genetic information into a digital code format;
System as per claim 1, involving method and system to convert numeric codes to alphabetical codes;
System as per claim 1, involving method and system of integrating genetic and non-genetic information for digital identification and personalization purposes;
System as per claim 1, involving method and system to decode digital identity code back to genetic information;
System as per claim 1, involving method and system to decode alphabetical code to numerical code;
System as per claim 1, involving method and system to convert digital identity code into other modes of identification;
System as per claim 1, involving method and system to register genetic based digital identification code to blockchain; and,
System as per claim 1, involving method and system to use the personalized digital identification code in blockchain based applications.
